(19) United States
(12) Patent Application Publication
(10) Pub. No.: US 2022/0031988 A1
(43) Pub. Date: Feb. 3, 2022

(54) Anti-virus bi-directional UV respirator

(71) Applicant: Sergey Anatoly KONOVALOV, Nesher (IL)

(72) Inventor: Sergey Anatoly KONOVALOV, Nesher (IL)

(21) Appl. No.: 16/945,875

(22) Filed: Aug. 2, 2020

(51) Int. Cl. A61M 16/06; A61L 9/20

(52) U.S. Cl. CPC A61M 16/0616; A61L 9/20; A61L 2209/12; A61M 2202/206; A61M 2202/203

(57) ABSTRACT

The present invention relates to health protection systems in general and to respiratory protection systems for both the user and the environment of his people from infections he himself has, having a headgear that a person wears and protects a person from infections and dangers transmitted by the air, and also purifies the air He exhales into the environment.

### BACKGROUND OF THE INVENTION

The present invention relates to health protection systems in general and to respiratory protection systems for both the user and the environment of his people from infections he himself has, having a headgear that a person wears and protects a person from infections and dangers transmitted by the air, and also purifies the air He exhales into the environment.

Airborne transmission of bacteria and viruses, mainly respiratory diseases. is a major public health problem. Controlling the spread of diseases transmitted by airborne droplets becomes more and more important during a pandemic influenza because COVID 19 is very dangerous for the human respiratory organs. This created the need for an inexpensive, effective, respiratory air disinfection system (air purification system).

### BRIEF DESCRIPTION OF THE DRAWINGS

FIG. 1 is schematic view of respirator frame. the upper part and the lower part of the frame are flat.

FIG. 2 is schematic view of respirator frame consists of several parallel narrower frames in which everything mentioned in terms of configuration and principle of operation is preserved.

### DETAILED DESCRIPTION OF THE INVENTION

The risk of airborne contamination can be reduced by protecting the respiratory system and eyes from airborne microorganisms and by providing clean air when exhaled.

Air purification can be achieved by killing infectious microorganisms with ultraviolet (UV) radiation. Ultraviolet radiation can be used as a screen inside a protective frame to destroy microorganisms in the air passing through this screen. Air movement through the screen is provided by inhalation or exhalation of the person himself, thus purifying him.

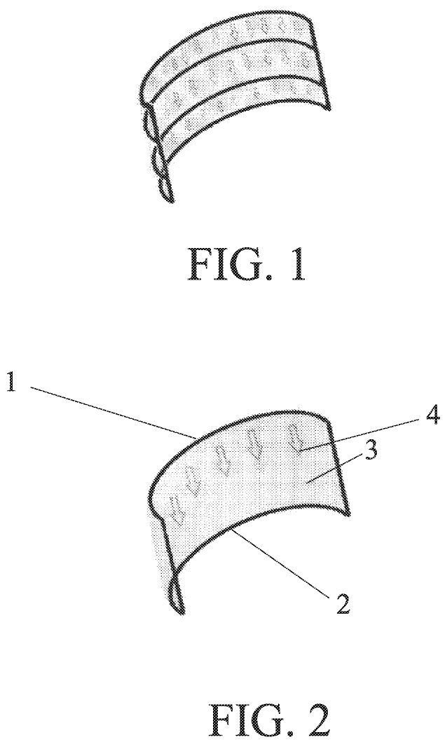

A rectangular, oval or other frame in which the inside of one side emits UVA, UVB and UVC light (or another type of radiation killing a virus) on the other side, creating a closed screen of UVA, UVB and UVC radiation. The frame (drawing 1. FIG. 1) is placed in front of the person's respiratory tract (similar to the protection of a medical mask) so that it disinfect the air entering and leaving the person's lungs. The frame can be fixed in different ways (for example, on the atop a visor; on glasses, etc.) at a safe fixed distance from the skin of a person so as not to be exposed to radiation.

The thickness of the frame should ensure maximum isolation of radiation in the plane of the frame so that the radiation does not go beyond its closed contour and does not fall on human skin. For this purpose, the frame can be divided into several similar narrower parallel frames (drawing 1. FIG. 2) with the same principle of operation and located in the outline of the general frame. This will reduce radiation scattering and increase the safety of use.

UV LEDs (preferably) located on the frame in one or more rows emit in the wavelength range of 240 nm to 280 nm and are powered by a battery connected to a voltage regulator. A current transformer is connected between the voltage regulator and the specified UV LEDs to supply them with a pulse current there is an option to use the solar cell to directly power the UV LEDs or to charge the battery, and the solar cell can be mounted on a visor attached to the indicated mask.

In this way, a person is protected from the absorption of viruses from the environment and disinfects the air escaping from a person's lungs. At the same time, it does not complicate breathing, which is present in a conventional medical mask. The mask also does not interfere with eating food without removing it.

FIG. 1 is schematic view of respirator frame. the upper part and the lower part of the frame are flat (FIG. 1 item 1 and 2) and they are parallel.

An upper part of the frame 1 contains a row or rows of UV-LED lights along the inner contour, that emits light to the opposite inner side (item 2) of a lower part of the frame 2. Lower part of the frame 2 is parallel to the upper part of the frame 1.

A direction of emitting light 3 from the upper part of the frame 1 and a flat UV screen 3 inside the frame.

FIG. 2 is schematic view of respirator frame consists of several parallel narrower frames in which everything mentioned in terms of configuration and principle of operation is preserved

1. A respirator frame comprising: is sized and configured to cover at least the user's nose and mouth; the upper part and the lower part of the frame are flat (FIG. 1 item 1 and 2) and they are parallel.

2. The respirator frame according to claim 1 with UV illumination, containing one or more rows of UV-LED or UV-DIOD lights along the inner contour of the upper part (element 1) emitting light (element 4) to the opposite inner side of the frame (element 2), crossing the air channel creating a continuous screen (element 3) between the top part (element 1) and lower part (element 2) of the frame.

3. The respirator frame of claim 1, wherein said plurality of ultraviolet LED lights and ultraviolet LED lamps all emit light in a wavelength range of 240 to 280 nm.

4. The respirator frame of claim 2, further comprising a battery or solar cell connected to a voltage regulator and a current chopping circuit connected between said voltage regulator and said plurality of UV LEDs to deliver pulsed current (standard configuration for its purpose).

5. A respirator frame according to claim 3, wherein said solar cell can be mounted on a visor attached to said mask.

6. The respirator of claim 4, wherein all of said plurality of UV LEDs emit light in a wavelength range of 240 to 280 nm.

7. The respirator frame of claim 4, wherein all of said plurality of UV light emitting diodes or LEDs emit light in a concentrated beam creating a flat UV screen (FIG. 1 item 3).

8. The respirator frame according to claim 1-5, consists of several parallel narrower frames (drawing 1. FIG. 2) in which everything mentioned in terms of configuration and principle of operation is preserved.
